(19) United States
(12) Patent Application Publication
(10) Pub. No.: US 2026/0091145 A1
(43) Pub. Date: Apr. 2, 2026

(54) CONTRAST AGENTS FOR ENHANCED MAGNETIC RESONANCE IMAGING

(71) Applicants: SAN JOSE STATE UNIVERSITY RESEARCH FOUNDATION, San Jose, CA (US); University of Redlands, Redlands, CA (US)

(72) Inventors: David Brook, San Jose, CA (US); David Paul Soulsby, Redlands, CA (US)

(21) Appl. No.: 19/345,871

(22) Filed: Sep. 30, 2025

Related U.S. Application Data
(60) Provisional application No. 63/700,901, filed on Sep. 30, 2024.

(51) Int. Cl. A61K 49/10; A61K 31/495; A61K 31/706; A61K 49/20; C07D 493/04; C07H 7/06; C07H 23/00

(52) U.S. Cl. CPC A61K 49/10; A61K 31/495; A61K 31/706; A61K 49/20; C07D 493/04; C07H 7/06; C07H 23/00

(57) ABSTRACT

The disclosure relates to compounds of the formula (Ia), (Ib), (IIa), and (IIb) and the use of the compounds of the formula (IIa) and (IIb) in method for imaging (e.g., magnetic resonance imaging) a subject in need thereof.

### CROSS-REFERENCE TO RELATED APPLICATIONS

This application claims the benefit of priority from U.S. Appl. No. 63/700,901, filed Sep. 30, 2024, which is incorporated by reference as if fully set forth herein.

### BACKGROUND

MRI scans often use paramagnetic chelated gadolinium-based contrast agents for image enhancement. This occurs through enhanced magnetic relaxation of biological fluids at the site of the contrast agent. However, gadolinium-based contrast agents can be toxic to patients with kidney disease, resulting in nephrogenic systemic fibrosis, a progressive thickening of skin and subcutaneous tissue. Patients must be carefully screened for signs of kidney disease prior to the use of a contrast agent, and this can limit the use of this important diagnostic tool. Alternative contrast agents that do not include toxic heavy metals are therefore needed.

### SUMMARY

The disclosure relates to a new class of sugar-derived biologically stable paramagnetic molecules useful as non-toxic MRI contrast agents. In contrast to existing MRI contrast agents, the sugar-derived biologically stable paramagnetic molecules described herein do not include any heavy metals and are therefore expected to be safer than current treatments. The sugar-derived structures of these molecules also allow for the targeting of specific disease-related biological structures.

More specifically, the disclosure relates to sugar-derived 6-oxoverdazyl free radical class of compounds. These compounds are expected to provide similar MRI enhancements as compared to the commercially available gadolinium-based systems but without any heavy metal toxicity concerns. Their structural tractability also means that they could be tailored to target specific biological systems allowing for image enhancement of that specific disease state.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The disclosure relates to compounds of the formula (Ia) or (Ib):

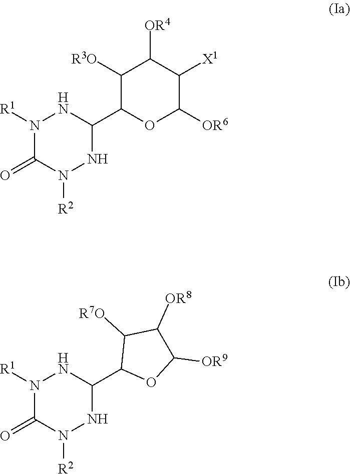

or a salt thereof,
wherein:
R¹ and R² are each independently alkyl, arylalkyl or aryl;
R³, R⁴, and R⁶ are each independently alkyl, a protecting group, a monosaccharide, a polysaccharide (e.g., a disaccharide, trisaccharide, and the like) or a polypeptide;
R⁷, R⁸, and R⁹ are each independently alkyl or R⁷ and R⁸ or R⁸ and R⁹, together with the oxygen atoms to which they are attached, form a heterocyclyl group; and
X¹ is OR⁵, wherein R⁵ is H, a protecting group, a monosaccharide, a polysaccharide (e.g., a disaccharide, trisaccharide, and the like), a polypeptide or NHR¹⁰, wherein R¹⁰ is H or acyl.

The disclosure also relates to stable free radicals/paramagnetic compounds such as compounds of the formula (IIa) or (IIb):

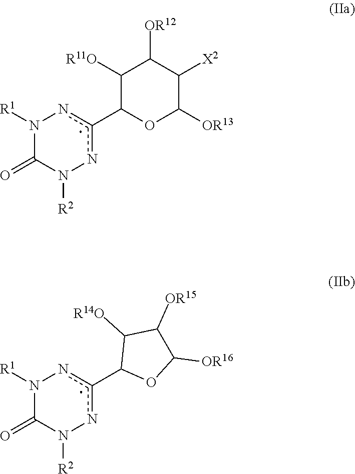

or a salt thereof,
wherein:
R¹ and R² are each independently alkyl, arylalkyl or aryl;
R¹¹R¹³ are each independently H, alkyl, a monosaccharide, a polysaccharide (e.g., a disaccharide, trisaccharide, and the like) or a polypeptide;
R¹⁴-R¹⁶ are each independently H, alkyl or R¹⁴ and R¹⁵ or R¹⁵ and R¹⁶, together with the oxygen atoms to which they are attached, form a heterocyclyl group; and
X² is OH or NHR¹⁰, wherein R¹⁰ is H or acyl.

In the compounds of the formula (Ia), R³ can be a protecting group. In embodiments, in the compounds of the formula (Ia), R⁴ can be a protecting group. In embodiments, in the compounds of the formula (Ia), R⁵ can be a protecting group. In embodiments, in the compounds of the formula (Ia), R⁶ can be a protecting group. In embodiments, in the compounds of the formula (Ib), R⁷ can be a protecting group. In embodiments, in the compounds of the formula (Ib), R⁸ can be a protecting group. In embodiments, in the compounds of the formula (Ib), R⁹ can be a protecting group. In embodiments, in the compounds of the formula (Ia), R³ and R⁴; R³ and R⁵; R³ and R⁶; R⁴ and R⁵; R⁴ and R⁶; R⁵ and R⁶; R³-R⁵; R³, R⁴, and R⁶; R⁴-R⁶; R³, R⁵, and R⁶; or R³-R⁶ can each independently be a protecting group. In embodiments, in the compounds of the formula (Ib), R⁷ and R⁸; R⁸ and R⁹; R⁷ and R⁹; or R⁷-R⁹ can each independently be a protecting group. In any of the foregoing, R³-R⁹ or R¹¹-R¹⁶ together with the oxygen atom to which each is attached can independently form a methoxymethyl ether, 2-methoxyethoxymethyl ether, tetrahydropyranyl ether, benzyl ether, p-methoxybenzyl ether, trimethylsilyl ether, triethylsilyl ether, triisopropyl silyl ether, t-butyldimethyl silyl ether, triphenylmethyl silyl ether, acetate ester, substituted acetate esters, pivaloate, benzoate, methanesulfonate and p-toluenesulfonate. In one embodiment, R³-R⁹ or R¹¹-R¹⁶ together with the oxygen atom to which each is attached can independently form a silyl ether, such as a trimethylsilyl ether.

In the compounds of the formula (Ia), (Ib), (IIa), and (IIb), R¹ and R² can each independently be (C₁-C₆)alkyl, such as (C₂-C₅)alkyl. In one example, R¹ or R² can be isopropyl. In another example, R¹ and R² are each isopropyl.

Additionally, or alternatively, X¹ can be OR⁵ in the compounds of the formula (Ia). Alternatively, in the compounds of the formula (Ia), X¹ can be NHR¹⁰, wherein R¹⁰ is acyl, such as alkylacyl (e.g., acetyl) or arylalkylacyl (e.g., benzoyl).

In the compounds of the formula (Ia), R⁶ can be alkyl, such as (C₁-C₆)alkyl or (C₂-C₅)alkyl. In one example, R⁶ can be methyl. Alternatively, R⁶ can be H.

In the compounds of the formula (Ib), R⁷ and R⁸ or R⁸ and R⁹, together with the oxygen atoms to which they are attached, form a heterocyclyl group. Thus, for example, in the compounds of the formula (Ib), R⁷ and R⁸ or R⁸ and R⁹, together with the oxygen atoms to which they are attached, form an acetonide of the formula:

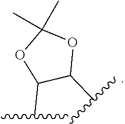 [SMILES: *C1OC(C)(C)OC1*]

In the compounds of the formula (IIa), X² can be OH. Alternatively, X² can be NHR¹⁰, wherein R¹⁰ is acyl, such as alkylacyl (e.g., acetyl) or arylalkylacyl (e.g., benzoyl). Additionally, or alternatively, R¹³ can be alkyl in the compounds of the formula (IIa), such as (C₁-C₆)alkyl or (C₂-C₅)alkyl. In one example, R¹³ can be methyl. Alternatively, R¹³ can be H.

In the compounds of the formula (IIb), R¹⁴ and R¹⁵ or R¹⁵ and R¹⁶, together with the oxygen atoms to which they are attached, form a heterocyclyl group. Thus, for example, in the compounds of the formula (IIb), R¹⁴ and R¹⁵ or R¹⁵ and R¹⁶, together with the oxygen atoms to which they are attached, form an acetonide of the formula:

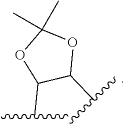 [SMILES: *C1OC(C)(C)OC1*]

Examples of the compounds of the formula (Ia) and (IIa) include compounds of the formula:

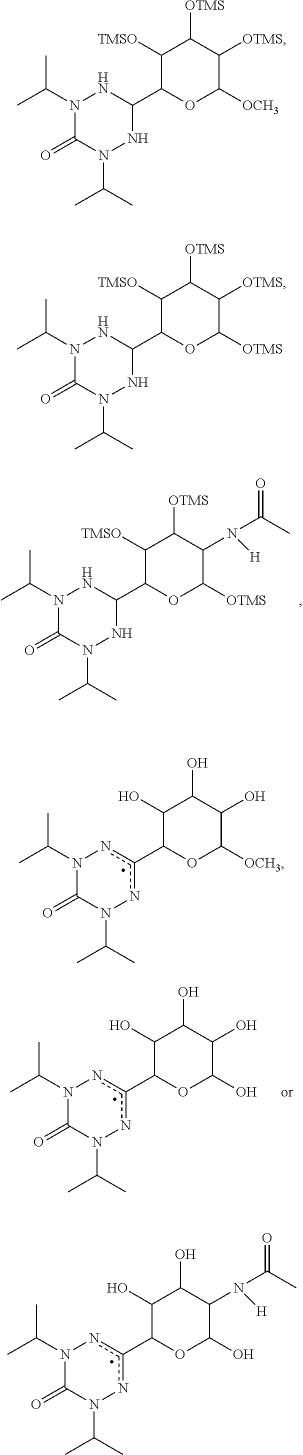

or a salt thereof and diastereomers thereof.

Examples of the compounds of the formula (Ia) and (IIa) include compounds of the formula:

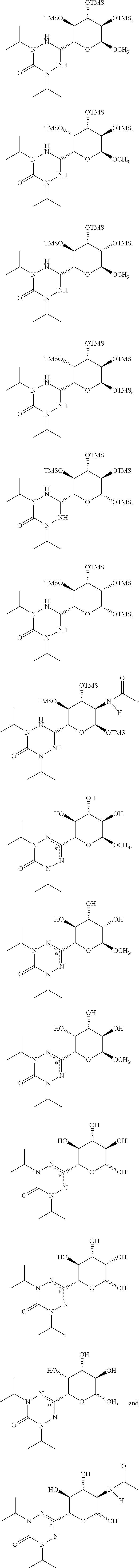

or salts thereof.

Examples of compounds of the formula (Ib) and (IIb) include compounds of the formula:

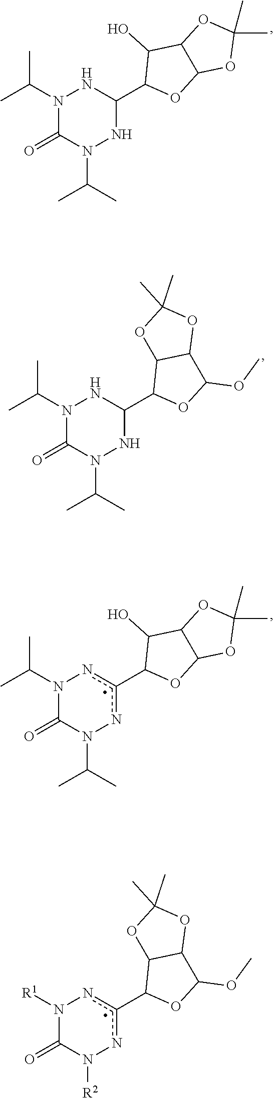

or a salt thereof and diastereomers thereof.

Examples of the compounds of the formula (Ib) and (IIb) include compounds of the formula:

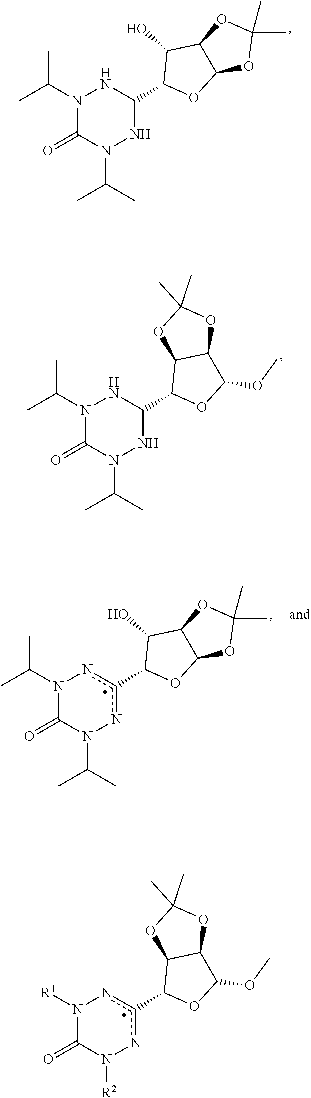

or salts thereof.

The disclosure also includes a method for magnetic resonance imaging, the method comprising:
administering at least one compound of the formula (IIa) and/or (IIb) to a subject; and
imaging the subject.

The subject can be any subject, such as a human subject or a veterinary subject (e.g., a canine or a feline). The at least one compound can be a compound of the formula:

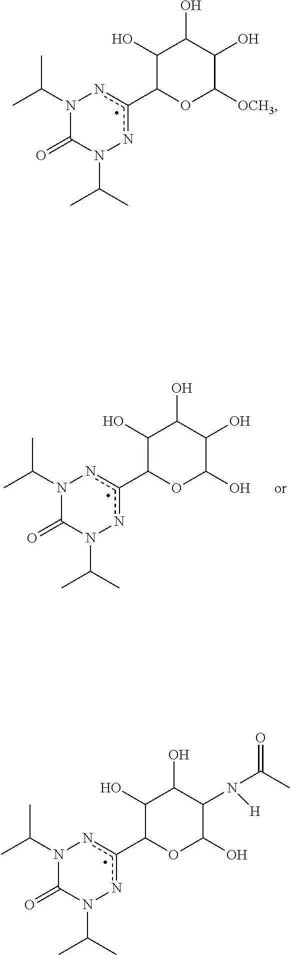

or a salt thereof; such as a compound of the formula:

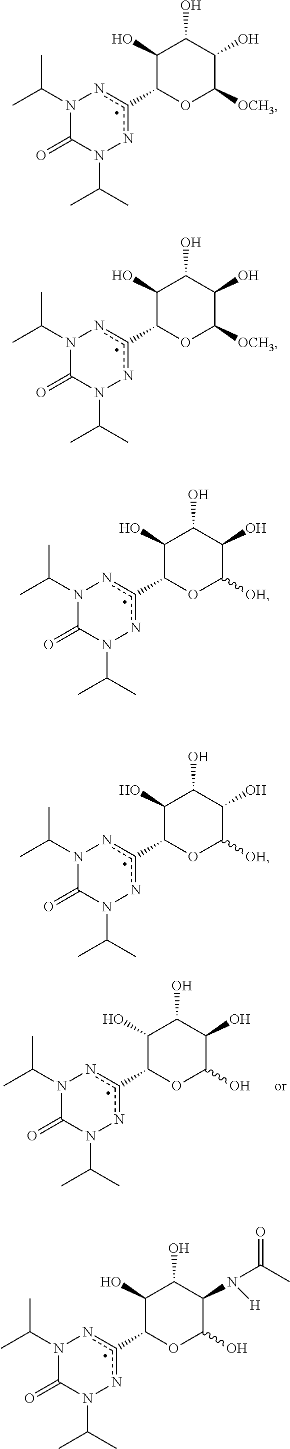

a salt thereof.

Any suitable route of administration can be used in the above methods, such as intravenous. An effective amount for, e.g., MRI, can be determined by one of ordinary skill in the art using dosage range determining methods known in the art. Typically, a physician (or veterinarian for non-human subjects) will determine the actual dosage, which will be most suitable for an individual subject. The specific dose level for an individual may be varied and will depend upon a variety of factors including the activity of the specific compound employed, the metabolic stability and length of action of that compound, the age, body weight, general health, gender, diet, mode and time of administration, rate of excretion, and other administered drugs.

The compounds of the formula (II) can be formulated as pharmaceutical compositions comprising a pharmaceutically acceptable carrier using methods well-known in the art. “Carrier” is used generically herein to refer to pharmaceutically acceptable carriers, diluents, adjuvants, and excipients. See, e.g., Remington: The Science and Practice of Pharmacy, 23rd edition, Oct. 30, 2020, Adeboye Adejare, ed. Accordingly, further provided is a pharmaceutical composition comprising the compound of formula (II) and a pharmaceutically acceptable carrier.

For oral administration, the compounds of the formula (II) can be formulated readily by combining them with pharmaceutically acceptable carriers well-known in the art. Such carriers enable the compounds to be formulated as tablets, pills, dragees, capsules, liquids, gels, syrups, slurries, suspensions and the like, for oral ingestion by a subject to be treated. Optionally the oral formulations can also be formulated in saline or buffers, e.g., EDTA for neutralizing internal acid conditions, or can be administered without any carriers.

The terms “substituted,” “substituent,” and “functional group” refer to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, —(CH₂)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

As used herein, the term “protecting group” refers to a group that, together with the oxygen atom to which it is attached (e.g., the oxygen atom to which each of R³-R⁹, and R¹¹-R¹⁶ is attached), forms a methoxymethyl ether, 2-methoxyethoxymethyl ether, tetrahydropyranyl ether, benzyl ether, p-methoxybenzyl ether, trimethylsilyl ether, triethylsilyl ether, triisopropyl silyl ether, t-butyldimethyl silyl ether, triphenylmethyl silyl ether, acetate ester, substituted acetate esters, pivaloate, benzoate, methanesulfonate and p-toluenesulfonate.

The term “alkyl” as used herein refers to substituted or unsubstituted straight chain and branched mono- or divalent alkyl groups and cycloalkyl groups having from 1 to 40 carbon atoms (C₁-C₄₀), 1 to 6 carbon atoms (C₁-C₆). 2 to 5 carbon atoms (C₂-C₅), 3 to 6 carbon atoms (C₃-C₆), 1 to about 20 carbon atoms (C₁-C₂₀), 1 to 12 carbons (C₁-C₁₂), 1 to 8 carbon atoms (C₁-C₅), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and ante-isoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched mono- or divalent alkenyl groups and cycloalkenyl groups having at least one double bond and having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkenyl groups include those with from 1 to 8 carbon atoms such as —CH═CH—, —CH═CHCH₃, and —CH₂CH═CHCH₂— groups, wherein the double bonds can have an E- or Z-configuration. And when there are multiple bonds, each double bond can, independently, have an E- or a Z-configuration. Examples of branched alkenyl groups include, but are not limited to, —CH═C(CH₃)— and CH₂C═CH(CH₃) groups. Representative substituted alkenyl groups can be substituted one or more times with any of the groups listed herein, for example, amino, hydroxy, cyano, carboxy, nitro, thio, alkoxy, and halogen groups.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups can have any number of carbon atoms, e.g., 3 to 8 carbon atoms (C₃-C₈), 3 to 6 carbon atoms (C₃-C₆), and 4 to 8 carbon atoms (C₄-C₈). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “cycloalkylalkyl” as used herein refers to substituted or unsubstituted alkyl groups as defined herein in which a hydrogen or carbon bond of an alkyl group as defined herein is replaced with a bond to a cycloalkyl group as defined herein. Representative cycloalkylalkyl groups include, but are not limited to, cyclopentylalkyl.

The term “alkylcycloalkyl” as used herein refers to substituted or unsubstituted cycloalkyl groups as defined herein in which a hydrogen of a cycloalkyl group as defined herein is replaced with a bond to an alkyl group as defined herein. Representative alkylcycloalkyl groups include, but are not limited to, alkylcyclopropyl.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group, as the term “heterocyclyl” is defined herein. An example of a heterocyclylcarbonyl group is a prolyl group, wherein the prolyl group can be a D- or an L-prolyl group.

The term “aryl” as used herein refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. “Aryl” and the phrase “aryl group” includes fused ring species including those that include fused aromatic and non-aromatic groups. Accordingly, “aryl” and the phrase “aryl group” include groups of the formula:

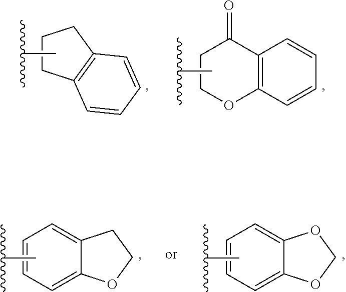

each of which can be substituted or unsubstituted, such as hydroxy substituted.

Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” or “heterocyclo” refers to substituted or unsubstituted aromatic and non-aromatic ring compounds containing 3 or more ring members, of which one or more (e.g., 1, 2 or 3) is a heteroatom such as, but not limited to, N, O, and S. Thus, a heterocyclyl can be a cycloheteroalkyl or a heteroaryl or, if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆), 3 to 5 carbon atoms (C₃-C₅) or 6 to 8 carbon atoms (C₆-C₈). A heterocyclyl group designated as a C₂-heterocyclyl can be a 5-ring with two carbon atoms and three heteroatoms, a 6-ring with two carbon atoms and four heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds, such as in the group 3,6-dihydro-2H-pyran and 3,4-dihydro-2H-pyran, having the formula:

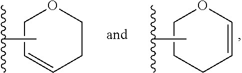

respectively, each of which can be substituted.

A heteroaryl ring is an embodiment of a heterocyclyl group. The phrase “heterocyclyl group” includes fused ring species including those that include fused aromatic and non-aromatic groups. Representative heterocyclyl groups include, but are not limited to tetrahydro-2H-thiopyran-1,1-dioxide, having the formula:

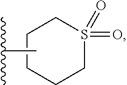

which can be substituted, 4a,5,6,7-tetrahydro-4H-pyrrolo[1,2-d][1,3,4]oxadiazinyl, having the formula:

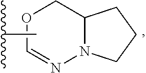

which can be substituted, pyrrolidinyl, pyrrolidinone (e.g., pyrrolidin-2-one), azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, imidazo[1,2-a]pyridinyl, having the formula:

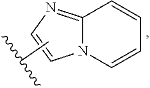

which can be substituted, triazyolyl, tetrazolyl, benzoxazolinyl, thiazolyl, benzthiazolinyl, and benzimidazolinyl groups. Examples of indolinonyl groups include groups having the general formula:

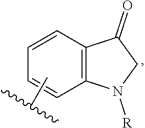

wherein R is as defined herein.

Examples of isoindolinonyl groups include groups having the general formula:

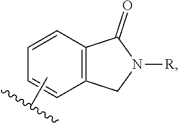

wherein R is as defined herein.

Examples of benzoxazolinyl groups include groups having the general formula:

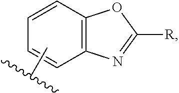

wherein R is as defined herein.

Examples of benzthiazolinyl groups include groups having the general formula:

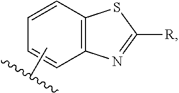

wherein R is as defined herein.

In some embodiments, the group R in benzoxazolinyl and benzthiazolinyl groups is an N(R)₂ group. In some embodiments, each R is hydrogen or alkyl, wherein the alkyl group is substituted or unsubstituted. In some embodiments, the alkyl group is substituted with a heterocyclyl group (e.g., with a pyrrolidinyl group).

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heterocyclylalkoxy” refers to alkyl groups as defined herein in which a hydrogen or carbon bond of an alkyl group as defined herein is replaced with a bond to a heterocyclyl group as defined herein and the alkyl group is attached to an oxygen. Representative heterocyclylalkoxy groups include, but are not limited to, —O—(CH₂)_{q} heterocyclyl, wherein q is an integer from 1 to 5. In some embodiments, heterocyclylalkoxy groups include —O—(CH₂)_{q} morpholinyl such as —O—CH₂CH₂-morpholine.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The terms “amine,” “amine group,” “amino,” and “amino group” refer to a substituent of the form —NH₂, —NHR, —NR₂, or —NR₃⁺, wherein each R is defined herein, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group.

An “alkylamino” group includes a monoalkylamino, dialkylamino, and trialkylamino group. An example of a “alkylamino” is —NH-alkyl and —N(alkyl)₂.

An example of a “cycloalkylamino” group is —NH-cycloalkyl and —N(cycloalkyl)₂.

An example of a “cycloalkyl heterocycloamino” group is —NH-(heterocyclo cycloalkyl), wherein the heterocyclo group is attached to the nitrogen and the cycloalkyl group is attached to the heterocyclo group.

An example of a “heterocyclo cycloamino” group is —NH-(cycloalkyl heterocycle), wherein the cycloalkyl group is attached to the nitrogen and the heterocyclo group is attached to the cycloalkyl group.

The term “amido” refers to a group of the formula —C(O)NR₂, wherein R is defined herein.

The terms “halo,” “halogen,” and “halide” group, by themselves or as part of another substituent, mean, unless otherwise stated, a fluorine, chlorine, bromine, or iodine atom.

The term “haloalkyl” group includes mono-halo alkyl groups, poly-halo alkyl groups, wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference.

In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

The disclosure also includes the following numbered Embodiments listed in no particular order of importance:

1. A compound of the formula (Ia) or (Ib):

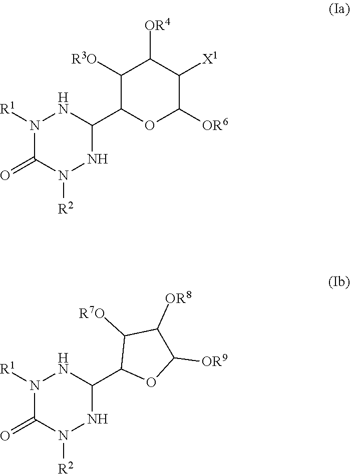

or a salt thereof,
wherein:
R¹ and R² are each independently alkyl, arylalkyl or aryl;
R³, R⁴, and R⁶ are each independently alkyl, a protecting group, a monosaccharide, a polysaccharide (e.g., a disaccharide, trisaccharide, and the like) or a polypeptide;
R⁷, R⁸, and R⁹ are each independently alkyl or R⁷ and R⁸ or R⁸ and R⁹, together with the oxygen atoms to which they are attached, form a heterocyclyl group; and
X¹ is OR⁵, wherein R⁵ is H, a protecting group, a monosaccharide, a polysaccharide (e.g., a disaccharide, trisaccharide, and the like), a polypeptide or NHR¹⁰, wherein
R¹⁰ is H or acyl.
   2. A compound of the formula (IIa) or (IIb):

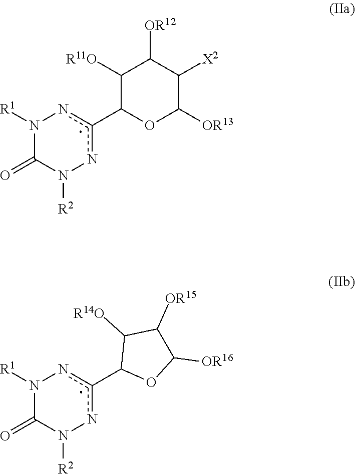

or a salt thereof,
wherein:
R¹ and R² are each independently alkyl, arylalkyl or aryl;
R¹¹R¹³ are each independently H, alkyl, a monosaccharide, a polysaccharide (e.g., a disaccharide, trisaccharide, and the like) or a polypeptide;
R¹⁴-R¹⁶ are each independently H, alkyl or R¹⁴ and R¹⁵ or R¹⁵ and R¹⁶, together with the oxygen atoms to which they are attached, form a heterocyclyl group; and
X² is OH or NHR¹⁰, wherein R¹⁰ is H or acyl.
   3. The compound of Embodiment 1 or 2, wherein R¹ and R² are each independently (C₁-C₆)alkyl.
   4. The compound of Embodiment 1 or 2, wherein R¹ and R² are each independently (C₂-C₅)alkyl.
   5. The compound of any preceding Embodiment, wherein R¹ or R² is isopropyl.
   6. The compound of any preceding Embodiment, wherein R¹ and R² are each isopropyl.
   7. The compound of any of Embodiments 1 and 3-6, wherein X¹ is OR⁵.
   8. The compound of any of Embodiments 1 and 3-6, wherein X¹ is NHR¹⁰.
   9. The compound of Embodiment 8, wherein R¹⁰ is alkylacyl or arylacyl.
   10. The compound of any of Embodiments 1 and 3-9, wherein R⁶ is alkyl.
   11. The compound of Embodiment 10, wherein R⁶ is (C₁-C₆)alkyl or (C₂-C₅)alkyl.
   12. The compound of Embodiment 10 or 11, wherein R⁶ is methyl.
   13. The compound of any of Embodiments 1 and 3-6, wherein R⁷ and R⁸ or R⁸ and R⁹, together with the oxygen atoms to which they are attached, form an acetonide of the formula:

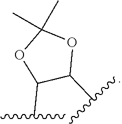 [SMILES: *C1OC(C)(C)OC1*]

14. The compound of any of Embodiments 2-6, wherein X² is OH.
15. The compound of any of Embodiments 2-6, wherein X² is NHR¹⁰.
16. The compound of Embodiment 15, wherein R¹⁰ is alkylacyl or arylalkylacyl.
17. The compound of any of Embodiments 2-6, 14, and 15, wherein R¹³ is alkyl.
18. The compound of Embodiment 17, wherein R¹³ is (C₁-C₆)alkyl or (C₂-C₅)alkyl.
19. The compound of any of Embodiments 2-6, 14, and 15, wherein R¹³ is H.
20. The compound of any of Embodiments 2-6 and 14-19, wherein R¹⁴ and R¹⁵ or R¹⁵ and R¹⁶, together with the oxygen atoms to which they are attached form an acetonide of the formula:

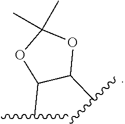 [SMILES: *C1OC(C)(C)OC1*]

21. The compound of any of Embodiments 1-13, wherein the compounds are of the formula:

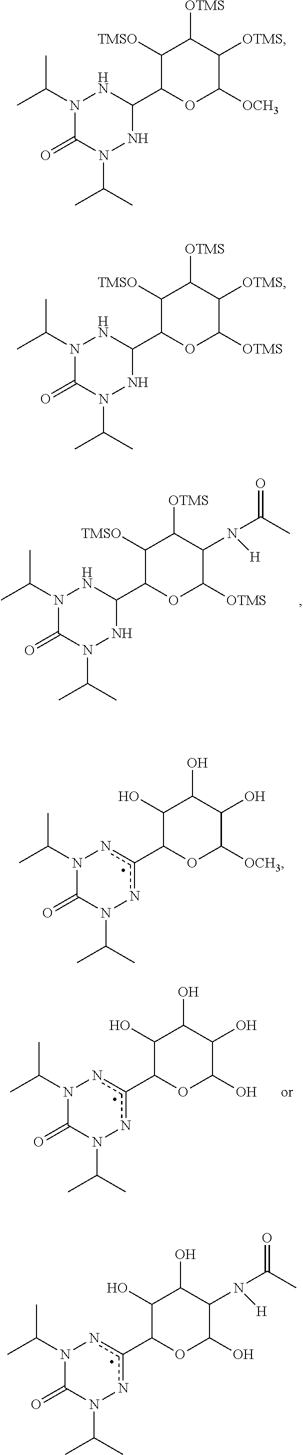

or a salt thereof and diastereomers thereof.
22. The compound of any of Embodiments 1-13 and 21, wherein the compounds are of the formula:

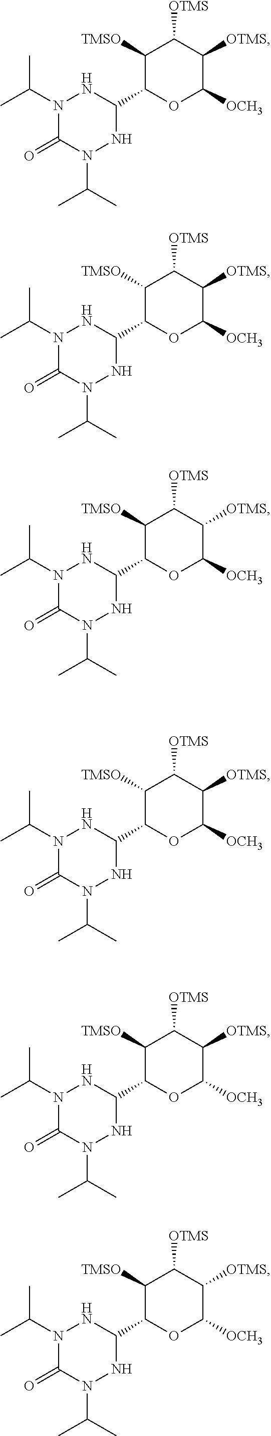

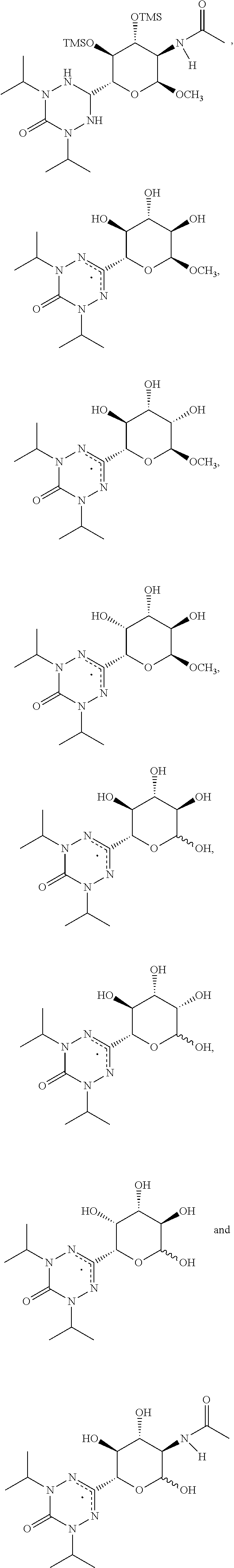

or salts thereof.
23. The compounds of any of Embodiments 2 and 14-20, wherein the compounds of the formula:

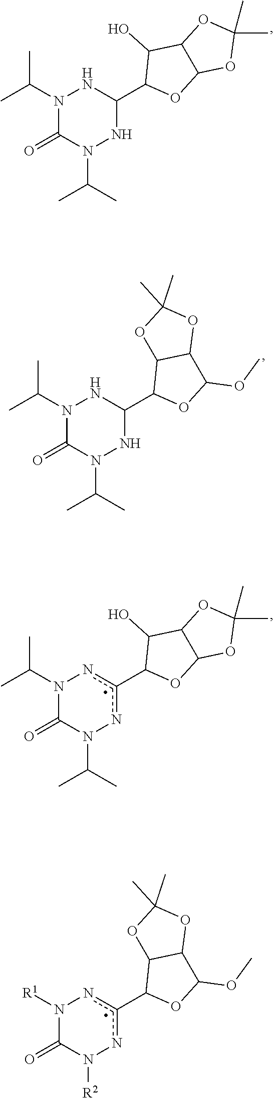

or a salt thereof and diastereomers thereof.
24. The compounds of any of Embodiments 2, 14-20, and 23, wherein the compounds of the formula:

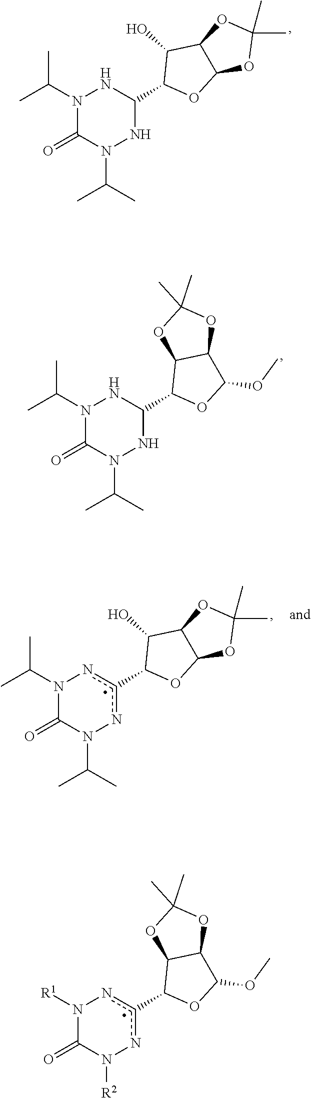

or salts thereof.
25. A pharmaceutical composition comprising at least one compound of any of Embodiments 1-24 and a pharmaceutically acceptable excipient.
26. A method for magnetic resonance imaging, the method comprising: administering at least one compound of any of Embodiments 1-24 or a pharmaceutical composition of Embodiment 25 to a subject; and imaging the subject.
27. The method of Embodiment 26, wherein the subject is a human subject or a veterinary subject.

### Examples

The disclosure can be better understood by reference to the following examples which are offered by way of illustration. The disclosure is not limited to the examples given herein.

### Experimental

### General Experimental

1D and 2D NMR spectra (¹H, ¹³C, COSY, DQCOSY, HSQC, HMBC) were recorded in deuterated solvents (CDCl₃ or D₂O) at 300 K using a Varian 400 MHz MR spectrometer. Chemical shifts are reported in parts per million (ppm, 5) NMR relative to residual CHCl₃ (7.26 ppm) for organic soluble compounds or 3-(trimethylsilyl)-1-propanesulfonic acid sodium salt (0.00 ppm) for aqueous soluble compounds. ¹H NMR splitting patterns are designated as singlet (s), doublet (d), triplet (t), doublet of doublets (dd), doublet of triplets (dt), multiplet (m), or broad (br). Coupling constants (J) are reported in Hertz (Hz). Signals caused by rotational hindrance are indicated by a star (*). All reactions were conducted in oven-dried glassware. Reactions were monitored by thin layer chromatography (TLC) on glass-backed plates (60 Å porosity silica gel, 250 μm thickness, with a 254 nm fluorescent indicator). Thin layer chromatography (TLC) plates were run using ratios of hexanes/ethyl acetate or dichloromethane/methanol as eluent and visualized using orcinol or potassium permanganate stains, or UV light. Isolation of compounds was carried out using flash chromatography (Biotage Isolera automated system visualizing at all wavelengths, 10 g silica gel cartridges—100 Å pore size, 60 μm). Chromatography methods are reported in column volumes (CVs). High-resolution mass spectrometry (HRMS) was obtained using an Agilent-6500 ESI TOF spectrometer with samples dissolved in CH₃CN. CDCl₃ was purchased from Cambridge Isotope Laboratories and stored over 4 Å molecular sieves. Solvents were purchased and used as received except where noted (companies in parentheses): Pentane (Acros Organics), hexanes (Pharmco), ethyl acetate (Pharmco), dichloromethane (VWR Chemicals), pyridine (Fisher Chemical) distilled over CaH₂ and stored over 4 Å molecular sieves, Dess Martin periodinane (Chem Scene), Poly(4-vinylpyridine), 25% crosslinked with divinylbenzene, ca. 18 mesh (Acros Organics).

### General Procedure for the Synthesis of C-6 Tetrazane TMS-Carbohydrates

To an oven-dried flask was charged the per-TMS-carbohydrate (1 equiv., 2 mmol), CH₂Cl₂ (10 mL), and pyridine (2 equiv., 4 mmol). With vigorous stirring, Dess-Martin periodinane (1.5 equiv., 3 mmol) was added in portions and the solution stirred for a further 1.5-2 hours during which time the solution goes from clear to opaque with the formation of a precipitate. [Analysis by TLC is challenging due to the sensitivity of the aldehyde. However, progress can be monitored via ¹H NMR spectroscopy using a small aliquot of the reaction. Completion of the oxidative deprotection is demonstrated by the complete disappearance of the anomeric hydrogen atom of the starting material, the appearance of a new anomeric hydrogen atom signal and an aldehyde signal.] After 1.5-2 hours, pentane (50 mL) is added to precipitate out the periodinane by-products. The organic layer is washed with water (3×50 mL) then dried with MgSO₄, filtered, and concentrated in vacuo to yield the crude aldehyde as a colorless oil.

Concurrently with the oxidative deprotection, 2,4-diisopropylcarbonohydrazide bis-hydrochloride (1.1 equiv., 2.2 mmol) is added to CH₂Cl₂ (10 mL). Poly(4-vinylpyridine) (mass=3×the mass of the hydrazide) and a small amount of MgSO₄ to remove residual water is added and this mixture is stirred for 1-2 hours. The crude aldehyde is dissolved in CH₂Cl₂ (5 mL), and the free hydrazide is filtered into the flask containing the aldehyde, with the PVP resin being washed with CH₂Cl₂ (10 mL). Stirring is continued for 1 h with the reaction typically turning a pale yellow during that time. The reaction is concentrated and chromatographed using a pentane/ethyl acetate gradient.

### General Procedure for the Deprotection and Formation of the 6-Oxoverdazyl Radical of C-6 Tetrazane TMS-Carbohydrates

Methyl glycosides (C-6 tetrazane TMS-OMe-carbohydrates) are dissolved in MeOH (10 mL) and Amberlyst-15 (two times the mass of the tetrazane) is added. The solution is stirred for 30 minutes, after which time the Amberlyst-15 is filtered off, washed with MeOH (10 mL), and the resulting solution concentrated to give the deprotected C-6 tetrazane carbohydrate in quantitative or near quantitative yield. For C-6 tetrazane TMS-OMe-carbohydrates that are not glycosides, the compound is dissolved in a 1:1 solvent mixture of water:acetontrile and the reaction is stirred for 12 hours. The deprotected C-6 tetrazane carbohydrate is dissolved in the minimum amount of water or water:MeOH for methyl glycosides (or less polar) derivatives. A second solution of K₃[Fe(CN)₆](3.1 equiv.) in H₂O (2 mL) with 2M Na₂CO₃ (15 drops) is prepared and stirred to ensure that the K₃[Fe(CN)₆] is fully dissolved. This is then added dropwise to the tetrazane solution. A small amount of effervescence occurs, and the solution turns a dark yellow or yellow/green. After 1 hour the solution is concentrated. The resulting solid/oil is vigorously stirred with CH₃CN (20-30 mL), a small amount of MgSO₄ is added, the solution is filtered and concentrated. The product is purified using column chromatography using a CH₂Cl₂/MeOH gradient to yield a yellow solid.

### Data Using General Procedure 1

### 6-[(2R,3R,4S,5R,6S)-6-methoxy-3,4,5-tris[(trimethylsilyl)oxy]oxan-2-yl]-2,4-bis(propan-2-yl)-1,2,4,5-tetrazinan-3-one (from TMS-methyl α-D-glucopyranoside) (1a)

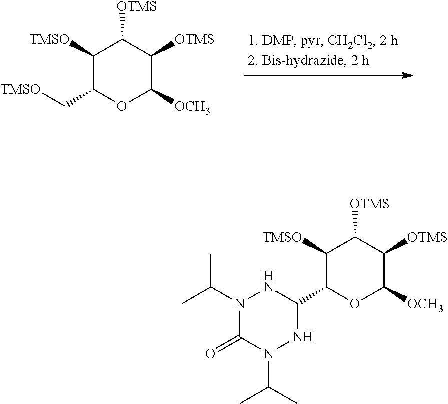

General Procedure 1. Chromatography conditions: [100:0 pentane (2 CV); 100:0 pentane/EtOAc to 90:10 pentane/EtOAc (15 CV); 90:10 pentane/EtOAc (15 CV)]. The product was recovered as a colorless solid (720 mg, 64%); Rf=0.39 (2:1 Hex/EtOAc). ¹H NMR (400 MHz, CDCl₃) δ 4.71 (septet, J=6.7 Hz, 1H), 4.63 (d, J=6.7 Hz, 1H), 4.60 (septet, J=6.7 Hz, 1H), 4.09-3.70 (m, 5H), 3.63 (m, 1H), 3.46 (dd, J=9.2, 3.5 Hz, 1H), 3.36 (s, 3H), 1.18 (d, J=6.7 Hz, 3H), 1.15 (d, J=6.7 Hz), 1.07 (d, J=6.7 Hz), 1.04 (d, J=6.7 Hz, 3H), 0.18 (s, 9H), 0.17 (s, 18H). ¹³C NMR (101 MHz, CDCl₃) δ 153.25, 99.78, 75.73, 73.52, 72.93, 70.46, 66.61, 54.98, 47.64, 47.15, 19.61, 19.55, 19.40, 18.32, 1.41 (3C), 1.11 (3C), 0.71 (3C). HRMS [M+H] Calc. 565.3267 Found. 565.3281.

### 6-[(2R,3R,4S,5S,6S)-6-methoxy-3,4,5-tris[(trimethylsilyl)oxy]oxan-2-yl]-2,4-bis(propan-2-yl)-1,2,4,5-tetrazinan-3-one (from TMS-methyl α-D-mannopyranoside) (1b)

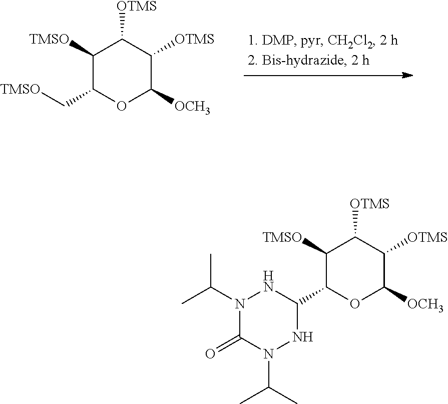

General Procedure 1. Chromatography conditions: [100:0 pentane (2 CV); 100:0 pentane/EtOAc to 90:10 pentane/EtOAc (15 CV); 90:10 pentane/EtOAc (15 CV)]. The product was recovered as a colorless solid (568 mg, 50%); Rf=0.43 (2:1 Hex/EtOAc).

¹H NMR (400 MHz, CDCl₃) δ 4.69 (septet, J=6.7 Hz, 1H), 4.62 (septet, J=6.7 Hz, 1H), 4.47 (s, 1H), 4.21 (d, J=12.3 Hz, 1H), 4.10 (t, J=9.1 Hz, 1H) 4.00 (br, 1H), 3.80-3.71 (m, 4H), 3.34 (s, 3H), 1.18 (d, J=6.7 Hz, 3H), 1.14 (d, J=6.7 Hz, 3H), 1.08 (d, J=6.7 Hz, 3H), 1.04 (d, J=6.7 Hz, 3H), 0.17 (s, 9H), 0.14 (s, 9H), 0.12 (s, 9H). ¹³C NMR (101 MHz, CDCl₃) δ 153.42, 101.71, 72.81, 72.57, 72.22, 69.09, 66.83, 54.90, 47.50, 47.15, 19.57, 19.50, 19.22, 18.31, 0.89 (3C), 0.54 (3C), 0.51 (3C). HRMS [M+H] Calc. 565.3267 Found. 565.3271.

### 2,4-bis(propan-2-yl)-6-[(2R,3R,4S,5R,6S)-3,4,5,6-tetrakis[(trimethylsilyl)oxy]oxan-2-yl]-1,2,4,5-tetrazinan-3-one (from β-TMS-glucopyranoside) (1d)

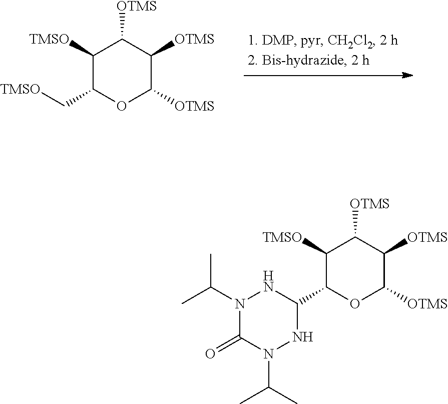

General Procedure 1. Chromatography conditions: [100:0 pentane (2 CV); 100:0 pentane/EtOAc to 90:10 pentane/EtOAc (15 CV); 90:10 pentane/EtOAc (15 CV)]. The product was recovered as a colorless solid (435 mg, 35%); Rf=0.59 (2:1 Hex/EtOAc). NMR appears to show hindered rotation and two conformations for one of the isopropyl groups. There looks to be a multiplet (probably septet) around 4.5 ppm (hidden beneath the doublet) that integrates to 0.5 ppm. This correlates by HSQC to the doublet at 1.5 ppm which integrates to 3. So, this would match one isopropyl group that has hindered rotation on the NMR timescale. These signals are labeled with a star below.

¹H NMR (400 MHz, CDCl₃) δ 4.68 (septet, J=6.7 Hz, 1H), 4.62 (septet, J=6.7 Hz, 1H), 4.49 (t, J=7.2 Hz, 1H), 4.47 (septet, J=6.7 Hz, 1H)*, 4.01-3.90 (m, 1H), 3.87-3.75 (m, 2H), 3.71-3.51 (m, 2H), 3.44 (t, J=8.4 Hz, 1H), 3.26 (t, J=8.0 Hz, 1H), 1.49 (d, J=7.0 Hz, 1H)*, 1.17 (d, J=6.7 Hz, 3H), 1.15 (d, J=6.7 Hz, 3H), 1.07 (d, J=6.7 Hz, 3H), 1.05 (d, J=6.7 Hz, 3H), 0.18 (d, J=1.0 Hz, 18H), 0.17 (s, 9H), 0.16 (s, 9H). ¹³C NMR (101 MHz, CDCl₃) δ 153.08, 98.45, 79.09, 77.34, 77.12, 77.02, 76.70, 75.25, 72.40, 66.38, 47.97*, 47.32, 47.22, 21.21*, 19.62, 19.55, 19.32, 18.37, 1.48 (3C), 1.43 (3C), 1.18 (3C), 0.58 (3C).

HRMS [M+H] Calc. 623.3506 Found. 623.3520.

### 2,4-bis(propan-2-yl)-6-[(2R,3S,4S,5R,6R)-3,4,5,6-tetrakis[(trimethylsilyl)oxy]oxan-2-yl]-1,2,4,5-tetrazinan-3-one (1e)

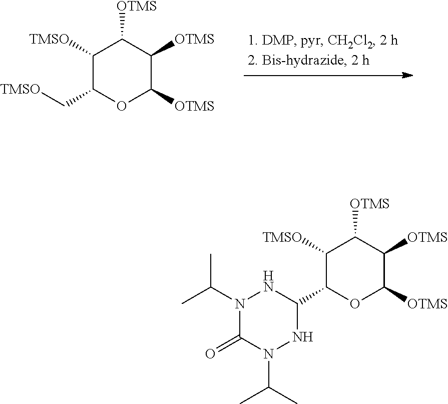

General Procedure 1. Chromatography conditions: [100:0 pentane (2 CV); 100:0 pentane/EtOAc to 90:10 pentane/EtOAc (15 CV); 90:10 pentane/EtOAc (15 CV)]. The product was recovered as a colorless solid (650 mg, 52%); Rf=0.52 (2:1 Hex/EtOAc). ¹H NMR (400 MHz, CDCl₃) δ 5.09 (d, J=3.1 Hz, 1H), 4.48 (septet, J=6.8 Hz, 1H), 4.46 (septet, J=6.8 Hz, 1H), 4.11-4.02 (m, 2H), 3.90-3.73 (m, 3H), 3.73 (br, 1H), 1.48 (d, J=6.7 Hz, 1H)*, 1.16-1.07 (m, 12H), 0.16 (s, 9H), 0.14 (s, 18H), 0.11 (s, 9H) [other NH not observed due to broadening]¹³C NMR (101 MHz, CDCl₃) δ 153.31, 94.67, 72.66, 70.44, 70.04*, 70.00, 69.46, 47.10, 47.08, * 46.56, 46.54, * 21.21, 19.90, 19.37, 19.22, 18.98, 0.90 (3C), 0.35 (3C), 0.33 (3C), 0.31 (3C). HRMS [M+H] Calc. 623.3506 Found. 623.3513.

### 2,4-bis(propan-2-yl)-6-[(2R,3R,4S,5S,6S)-3,4,5,6-tetrakis[(trimethylsilyl)oxy]oxan-2-yl]-1,2,4,5-tetrazinan-3-one (from TMS-mannopyranoside) (1f)

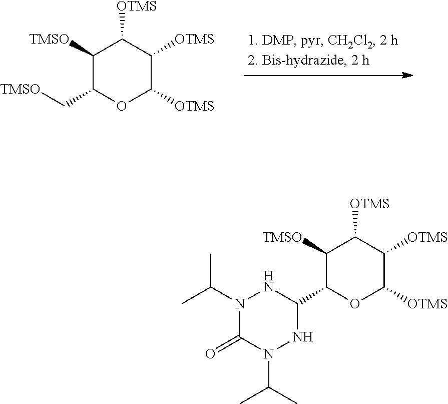

General Procedure 1. Chromatography conditions: [100:0 pentane (2 CV); 100:0 pentane/EtOAc to 90:10 pentane/EtOAc (15 CV); 90:10 pentane/EtOAc (15 CV)]. The product was recovered as a colorless solid (643 mg, 50%)

¹H NMR (400 MHz, CDCl₃) δ 4.88 (s, 1H), 4.68 (septet, J=6.6 Hz, 1H), 4.60 (septet, J=6.6 Hz, 1H), 4.18-4.07 (m, 2H), 3.90 (br, 1H), 3.87 (d, J=2.8 Hz, 2H), 3.73 (t, J=11.8 Hz, 1H), 3.65 (s, 1H), 1.17 (d, J=6.6 Hz, 3H), 1.14 (d, J=6.6 Hz, 3H), 1.06 (d, J=6.6 Hz, 3H), 1.03 (d, J=6.6 Hz, 3H), 0.18 (s, 9H), 0.17 (s, 9H), 0.15 (s, 9H), 0.12 (s, 9H). ¹³C NMR (101 MHz, CDCl₃) δ 153.26, 95.42, 74.36, 72.86, 71.64, 69.08, 66.68, 47.39, 47.11, 19.59, 19.48, 19.23, 18.31, 0.92 (3C), 0.53 (3C), 0.48 (3C), −0.10 (3C).

### N-[(2R,3R,4R,5R,6R)-6-[6-oxo-1,5-bis(propan-2-yl)-1,2,4,5-tetrazinan-3-yl]-2,4,5-tris[(trimethylsilyl)oxy]oxan-3-yl]acetamide (1 g)

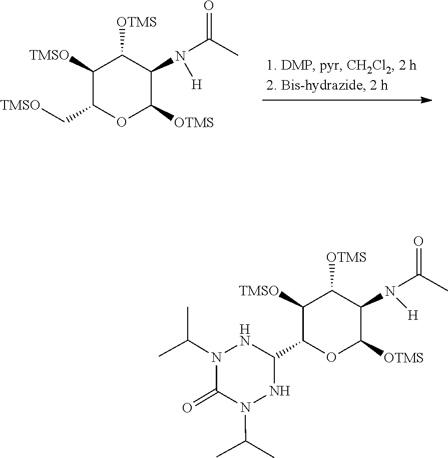

General Procedure 1. Chromatography conditions: [100:0 pentane (2 CV); 100:0 pentane/EtOAc to 60:40 pentane/EtOAc (15 CV); 60:40 pentane/EtOAc (15 CV)]. The product was recovered as a colorless solid (352 mg, 30%); Rf=0.29 (2:1 Hex/EtOAc). ¹HNMR (400 MHz, CDCl₃) δ 5.34-5.21 (m, 1H), 5.07 (d, J=3.3 Hz, 1H), 4.70 (septet, J=6.7 Hz, 1H), 4.61 (septet, J=6.7 Hz, 1H), 4.07-3.88 (m, 3H), 3.75 (m, 4H), 2.02 (s, 3H), 1.17 (d, J=6.7 Hz, 3H), 1.15 (d, J=6.7 Hz, 3H), 1.05 (d, J=6.7 Hz, 3H), 1.03 (d, J=6.7 Hz, 3H), 0.19 (s, 9H), 0.18 (s, 9H), 0.16 (s, 9H). ¹³C NMR (101 MHz, CDCl₃) δ 170.03, 152.99, 92.44, 73.74, 73.02, 70.98, 66.49, 54.17, 47.47, 47.15, 23.71, 19.68, 19.50, 19.46, 18.32, 1.24 (3C), 1.06 (3C), −0.09 (3C). HRMS [M+H] Calc. 592.3376 Found. 592.3385

### Data Using General Procedure 2

### 3-oxo-2,4-bis(propan-2-yl)-6-[(2R,3S,4S,5R,6S)-3,4,5-trihydroxy-6-methoxytetrahydro-2H-pyran-2-yl]-3,4-dihydro-1,2,4,5-tetraazin-1 (2H)-yl (2a)

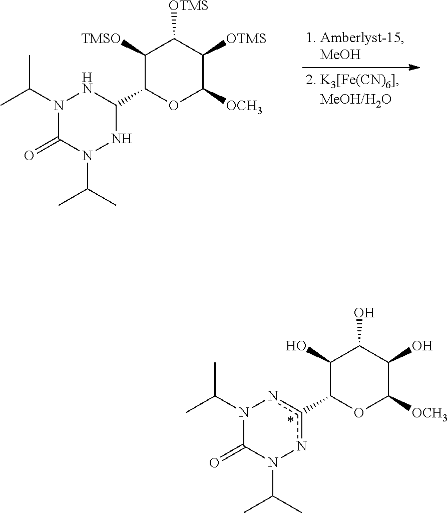

General Procedure 2 and 3. TMS-tetrazane (720 mg, 1.274 mmol). Radical chromatography conditions: [100:0 CH₂Cl₂ (2 CV); 100:0 CH₂Cl₂/MeOH to 60:40 CH₂Cl₂/MeOH (15 CV); 60:40 CH₂Cl₂/MeOH (15 CV)]. The product was recovered as a yellow solid (133 mg, 30%); Rf=0.34 (9:1 CH₂Cl₂/MeOH). HRMS [M] Calc. 345.1774 Found. 345.1775.

### 3-oxo-2,4-bis(propan-2-yl)-6-[(2R,3S,4S,5S,6S)-3,4,5-trihydroxy-6-methoxytetrahydro-2H-pyran-2-yl]-3,4-dihydro-1,2,4,5-tetraazin-1 (2H)-yl (2b)

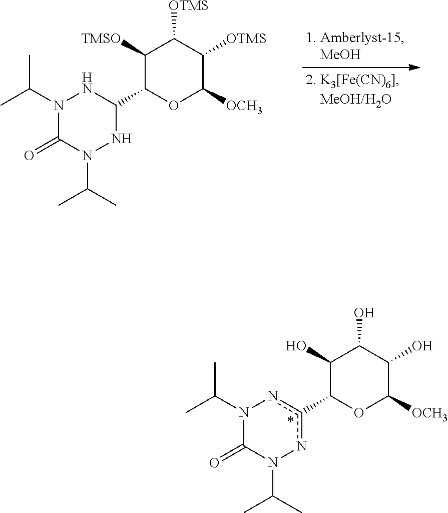

General Procedure 2 and 3. TMS-tetrazane (568 mg, 1.005 mmol). Radical chromatography conditions: [100:0 CH₂Cl₂ (2 CV); 100:0 CH₂Cl₂/MeOH to 60:40 CH₂Cl₂/MeOH (15 CV); 60:40 CH₂Cl₂/MeOH (15 CV)]. The product was recovered as a yellow solid (147 mg, 41%); Rf=0.31 (9:1 CH₂Cl₂/MeOH). HRMS [M] Calc. 345.1774 Found. 345.1773.

### 3-oxo-2,4-bis(propan-2-yl)-6-[(2R,3S,4S,5R)-3,4,5,6-tetrahydroxytetrahydro-2H-pyran-2-yl]-3,4-dihydro-1,2,4,5-tetraazin-1 (2H)-yl (2c)

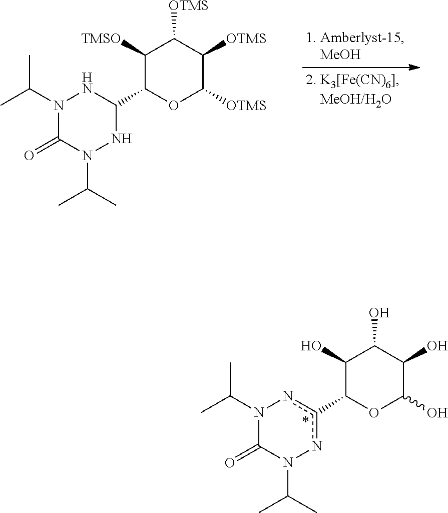

General Procedure 2. TMS-tetrazane (435 mg, 0.698 mmol). Chromatography conditions: [100:0 CH₂Cl₂ (2 CV); 100:0 CH₂Cl₂/MeOH to 60:40 CH₂Cl₂/MeOH (15 CV); 60:40 CH₂Cl₂/MeOH (15 CV)]. The product was recovered as a yellow solid (80 mg, 35%); Rf=0.20 (9:1 CH₂Cl₂/MeOH). HRMS [M] Calc. 331.1618 Found. 331.1636.

### 3-oxo-2,4-bis(propan-2-yl)-6-[(2R,3R,4S,5R)-3,4,5,6-tetrahydroxytetrahydro-2H-pyran-2-yl]-3,4-dihydro-1,2,4,5-tetraazin-1 (2H)-yl (2d)

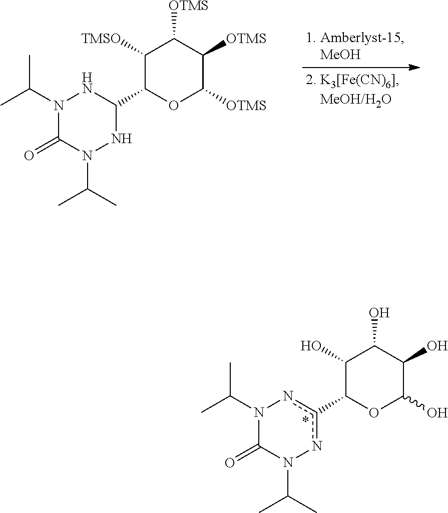

General Procedure 2. TMS-tetrazane (650 mg, 1.043 mmol). Chromatography conditions: [100:0 CH₂Cl₂ (2 CV); 100:0 CH₂Cl₂/MeOH to 50:50 CH₂Cl₂/MeOH (15 CV); 50:50 CH₂Cl₂/MeOH (15 CV)]. The product was recovered as a yellow solid (72 mg, 21%); Rf=0.63 (4:1 CH₂Cl₂/MeOH). HRMS [M] Calc. 331.1618 Found. 331.1628.

### 3-oxo-2,4-bis(propan-2-yl)-6-[(2R,3S,4R,5R)-3,4,6-trihydroxy-5-(methylcarbonylamino)tetrahydro-2H-pyran-2-yl]-3,4-dihydro-1,2,4,5-tetraazin-1 (2H)-yl (2e)

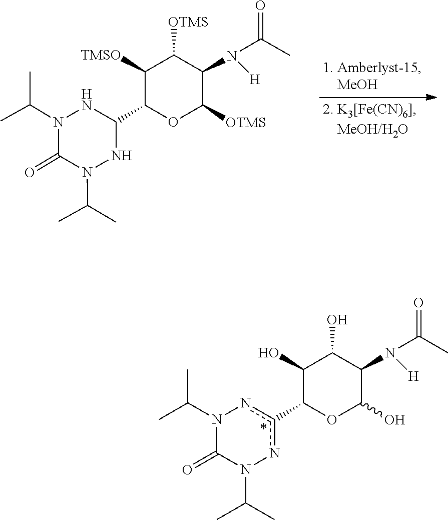

General Procedure 2. TMS-tetrazane (353 mg, 0.596 mmol). Chromatography conditions: [100:0 CH₂Cl₂ (2 CV); 100:0 CH₂Cl₂/MeOH to 50:50 CH₂Cl₂/MeOH (15 CV); 50:50 CH₂Cl₂/MeOH (15 CV)]. The product was recovered as a yellow solid (135 mg, 60%); Rf=0.45 (4:1 CH₂Cl₂/MeOH). HRMS [M+H] Calc. 373.1956 Found. 373.1982.

What is claimed is:

1. A compound of the formula (IIa), (IIb), (Ia) or (Ib): 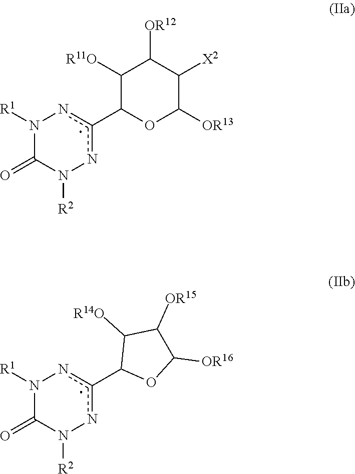
or a salt thereof,
wherein:
R¹ and R² are each independently alkyl, arylalkyl or aryl;
R¹¹-R¹³ are each independently H, alkyl, a monosaccharide, a polysaccharide (e.g., a disaccharide, trisaccharide, and the like) or a polypeptide;
R¹⁴-R¹⁶ are each independently H, alkyl or R¹⁴ and R¹⁵ or R¹⁵ and R¹⁶, together with the oxygen atoms to which they are attached, form a heterocyclyl group; and
X² is OH or NHR¹⁰, wherein R¹⁰ is H or acyl;
or 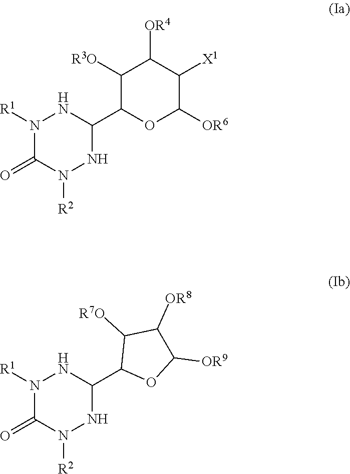
or a salt thereof,
wherein:
R¹ and R² are each independently alkyl, arylalkyl or aryl;
R³, R⁴, and R⁶ are each independently alkyl, a protecting group, a monosaccharide, a polysaccharide (e.g., a disaccharide, trisaccharide, and the like) or a polypeptide;
R⁷, R⁸, and R⁹ are each independently alkyl or R⁷ and R⁸ or R⁸ and R⁹, together with the oxygen atoms to which they are attached, form a heterocyclyl group; and
X¹ is OR⁵, wherein R⁵ is H, a protecting group, a monosaccharide, a polysaccharide (e.g., a disaccharide, trisaccharide, and the like), a polypeptide or NHR¹⁰, wherein R¹⁰ is H or acyl;

2. The compound of claim 1, wherein R¹ and R² are each independently (C₁-C₆)alkyl.

3. The compound of claim 1, wherein R¹ and R² are each isopropyl.

4. The compound of claim 1, wherein X¹ is OR⁵ or NHR¹⁰.

5. The compound of claim 4, wherein R¹⁰ is alkylacyl or arylacyl.

6. The compound of claim 1, wherein R⁶ is alkyl.

7. The compound of claim 6, wherein R⁶ is methyl.

8. The compound of claim 1, wherein R⁷ and R⁸ or R⁸ and R⁹, together with the oxygen atoms to which they are attached, form an acetonide of the formula:

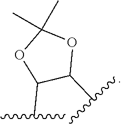 [SMILES: *C1OC(C)(C)OC1*]

9. The compound of claim 1, wherein X² is OH or NHR¹⁰.

10. The compound of claim 9, wherein R¹⁰ is alkylacyl or arylalkylacyl.

11. The compound of claim 1, wherein R¹³ is alkyl.

12. The compound of claim 1, wherein R¹³ is H.

13. The compound of claim 1, wherein R¹⁴ and R¹⁵ or R¹⁵ and R¹⁶, together with the oxygen atoms to which they are attached form an acetonide of the formula:

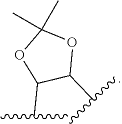 [SMILES: *C1OC(C)(C)OC1*]

14. The compound of claim 1, wherein the compounds are of the formula: 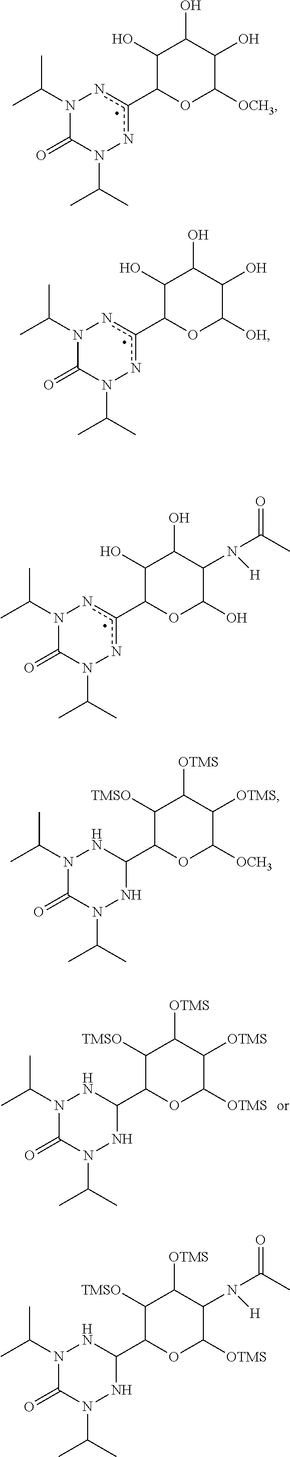
or a salt thereof and diastereomers thereof.

15. The compound of claim 1, wherein the compounds are of the formula: 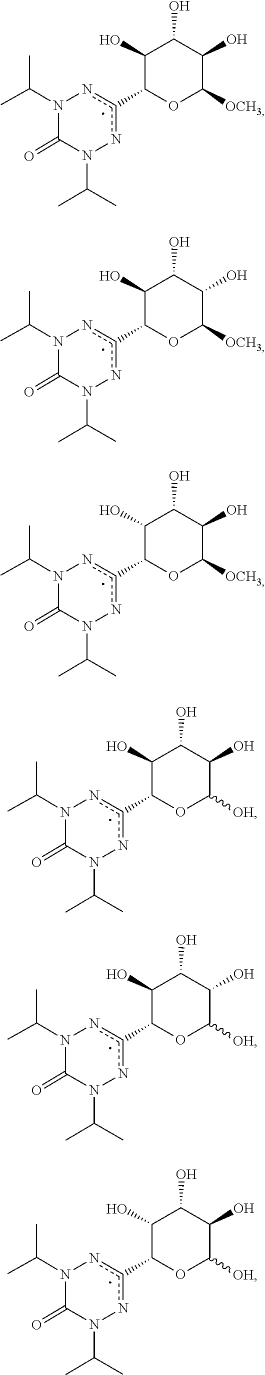 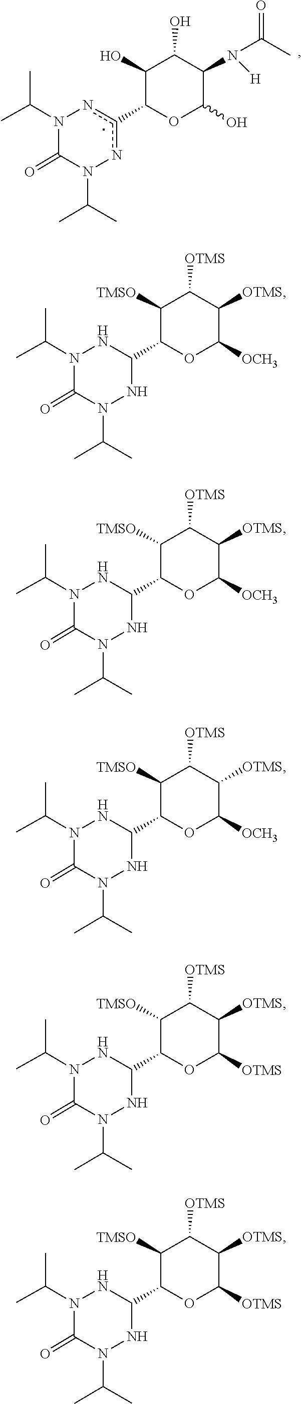 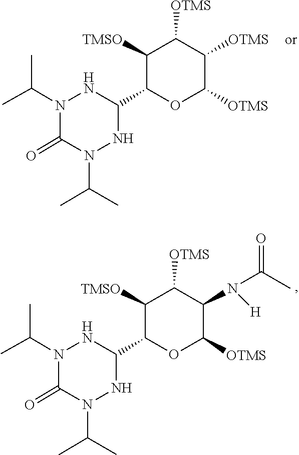
or a salt thereof and diastereomers thereof.

16. The compound of claim 1, wherein the compounds of the formula: 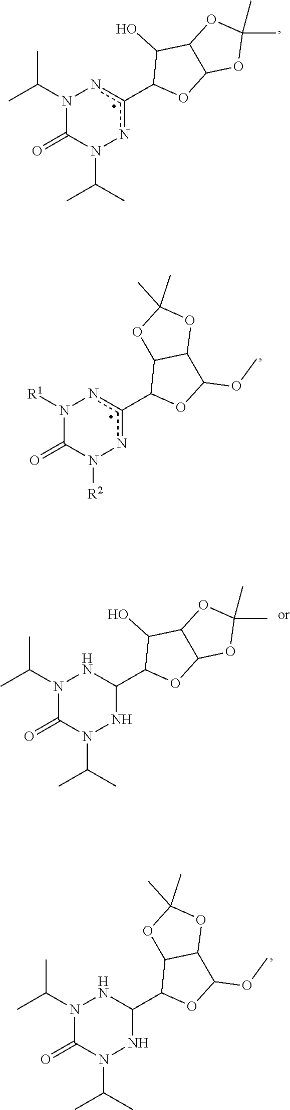
or a salt thereof and diastereomers thereof.

17. The compound of claim 1, wherein the compounds of the formula: 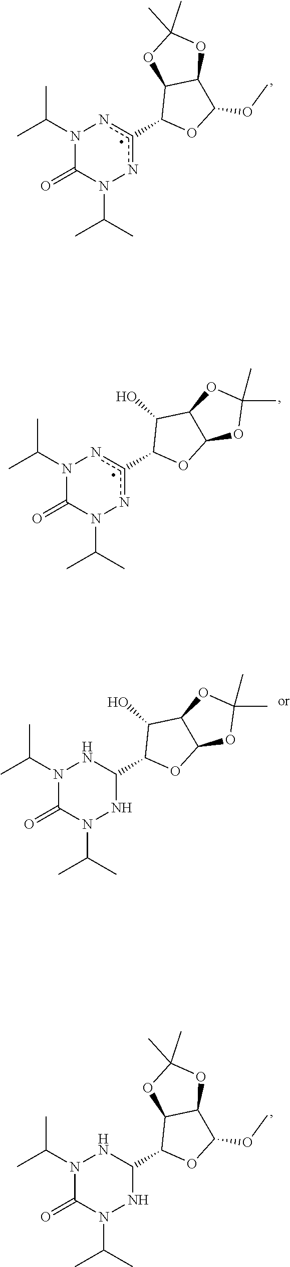
or a salt thereof and diastereomers thereof.

18. A pharmaceutical composition comprising at least one compound of claim 1 and a pharmaceutically acceptable excipient.

19. A method for magnetic resonance imaging, the method comprising:
administering at least one compound of claim 1 to a subject; and
imaging the subject.

20. The method of claim 19, wherein the subject is a human subject or a veterinary subject.
